SEQUENCE LISTING (10 sequences)

SEQ ID NO: 1 - DNA, 26 bp, Artificial Sequence
  Forward primer for the amplification of FMO3 gene
tgctgcagaactcagccatgtagctc

SEQ ID NO: 2 - DNA, 23 bp, Artificial Sequence
  Reverse primer for the amplification of FMO3 gene
atctgccttgtgtaccaccagtc

SEQ ID NO: 3 - DNA, 17 bp, Artificial Sequence
  Forward Primer for the amplification of TNF-alpha gene
agtgacaagcctgtagc

SEQ ID NO: 4 - DNA, 17 bp, Artificial Sequence
  Reverse primer for the amplification of TNF-alpha gene
tgaggagcacgtagtcg

SEQ ID NO: 5 - DNA, 20 bp, Artificial Sequence
  Forward primer for the amplification of VCAM-1 gene
agttggggattcggttgttc

SEQ ID NO: 6 - DNA, 20 bp, Artificial Sequence
  Reverse primer for the amplification of VACM-1 gene
cattccttaccaccccattg

SEQ ID NO: 7 - DNA, 18 bp, Artificial Sequence
  Forward primer for the amplification of E-selectin gene
ccagaatggcgtcatgga

SEQ ID NO: 8 - DNA, 20 bp, Artificial Sequence
  Reverse Primer for the amplification of E-selectin gene
taaagccctcattgcattga

SEQ ID NO: 9 - DNA, 18 bp, Artificial Sequence
  Forward primer for the amplification of GAPDH gene
tcaacggcacagtcaagg

SEQ ID NO: 10 - DNA, 19 bp, Artificial Sequence
  Reverse primer for the amplification of GAPDH gene
actccacgacatactcagc

(19) United States
(12) Patent Application Publication
(10) Pub. No.: US 2021/0093582 A1
(43) Pub. Date: Apr. 1, 2021

(54) COMPOSITIONS FOR REDUCING TRIMETHYLAMINE-N-OXIDE AND RELATED THERAPEUTIC APPLICATIONS

(71) Applicants: Muhammed Majeed, Bangalore (IN); Kalyanam Nagabhushanam, East Windsor, NJ (US)

(72) Inventors: Muhammed Majeed, Bangalore (IN); Kalyanam Nagabhushanam, East Windsor, NJ (US)

(21) Appl. No.: 16/585,181

(22) Filed: Sep. 27, 2019

(51) Int. Cl. A61K 31/09

(52) U.S. Cl. CPC A61K 31/09

(57) ABSTRACT

The present invention discloses a method for reducing the elevated levels of circulating Trimethylamine-N-oxide in mammals, using a composition comprising pterostilbene by modifying gut microbial diversity altered by foods rich in quaternary amines like choline, carnitine, glycine betaine (GBT), and phosphatidylcholine and lecithin and by inhibiting liver mono-oxygenase enzyme FMO3.

### BACKGROUND OF THE INVENTION

### Field of the Invention

The invention in general relates to pterostilbene compositions. More specifically, the present invention discloses compositions comprising pterostilbene for the reduction of trimethylamine-N-oxide by modifying gut microbial diversity and inhibiting liver mono-oxygenase enzyme.

### Description of Prior Art

Trimethylamine-N-oxide (TMAO), a by-product of gut microbial metabolism of L-carnitine, has been implicated in the development of many diseases and dysfunctions, particularly vascular inflammation. The effect of TMAO on cardiovascular health has been a focus of intense research recently. TMAO has been demonstrated to contribute to atherosclerosis and is highly associated with cardiovascular disease (CVD) risk (Tang, et al., “Intestinal Microbial Metabolism of Phosphatidylcholine and Cardiovascular Risk” New Engl. J Med. 2013 368, 1575-1584; Wang et al., “Gut flora metabolism of phosphatidylcholine promotes cardiovascular disease” Nature. 2011 472, 57-63). TMAO is also predicted to play a role in development of cancer (Lim et al., “TMAO, the Gut Microbiome, and Colorectal Cancer risk in the Multiethnic Cohort” NIH Project No. 5R01CA204368-02).

Foods (e.g. Red meat) rich in quaternary amines like choline (STR#1), L-carnitine (STR#2), glycine betaine (STR#3), phosphatidylcholine (STR#4) and lecithin have been reported to release TMAO by the metabolism of the above compounds by gut microbiota to form trimethylamine (TMA)—represented by STR#5 which is subsequently converted to trimethylamine-N-oxide (TMAO)—represented by STR#6, by host hepatic enzyme, flavin, monooxygenase 3 (FMO3) (Koeth et al., “Intestinal microbiota metabolism of L-carnitine, a nutrient in red meat, promotes atherosclerosis” Nat Med. 2013 19, 576-585: Randrianarisoa et al., “Relationship of Serum Trimethylamine N-Oxide (TMAO) Levels with early Atherosclerosis in Humans” Scientific Reports. 2016 6, 26745). Lecithins are mixtures of glycerophospholipids including phosphatidylcholine, phosphatidylethanolamine, phosphatidylinositol, phosphatidylserine, and phosphatidic acid. All the above compounds have a general trimethylamine structure represented by STR#5, which is responsible for the formation of TMAO.

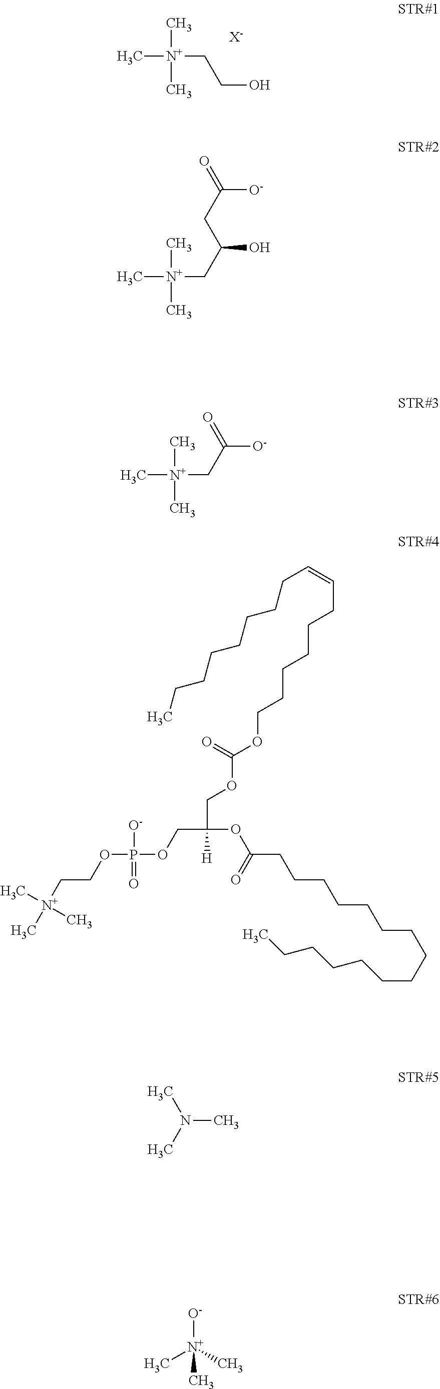

Although it is known that gut microbiota is responsible for the formation of TMAO, the genetic and biochemical mechanisms that are involved in TMAO formation remains unclear. Reports indicate that the first enzyme involved in carnitine metabolism as a two-component Rieske-type oxygenase/reductase, designated as CntA/B (Zhu et al., “Carnitine metabolism to trimethylamine by an unusual Rieske-type oxygenase from human microbiota” Proc Natl Acad Sci USA. 2014 111, 4268-4273). The genes encoding CntA/B were mainly observed in Proteobacteria, especially Gamma proteobacteria (mostly derived from Escherichia and Acinetobacter) and a few Beta proteobacteria and Firmicutes (Zhu et al., “Carnitine metabolism to trimethylamine by an unusual Rieske-type oxygenase from human microbiota” Proc Natl Acad Sci USA. 2014 111, 4268-4273; Rath et al., “Uncovering the trimethylamine producing bacteria of the human gut microbiota” Microbiome. 2017 5, 54)

Antibiotics that modify the gut microbial diversity markedly decreased TMAO levels (Koeth et al., “Intestinal microbiota metabolism of L-carnitine, a nutrient in red meat, promotes atherosclerosis” Nat Med. 2013 19, 576-585). However, antibiotics can cause many undesirable side effects and the concerns of antibiotic resistance issues. Recently studies have focused on the alternative strategies to reduce the elevation in plasma TMAO using natural molecules from plant sources. Pterostilbene, a natural dimethoxylated analog of resveratrol, has recently received tremendous attention. Pterostilbene was found to be more metabolically stable and exhibited stronger pharmacological activities than that of resveratrol (Wang et al., “Metabolism and pharmacokinetics of resveratrol and pterostilbene” BioFactors 2018 44, 16-25; Lee et al., “Chemoprevention by resveratrol and pterostilbene: Targeting on epigenetic regulation” BioFactors. 2018 44, 26-35). Pterostilbene exhibits numerous preventive and therapeutic properties in a vast range of human diseases that include neurological, cardiovascular, metabolic, and hematologic disorders and is generally considered to be safe for human consumption. Pterostilbene is also reported to modify gut microbial diversity, specifically bacterial species belonging to genera Akkermansia and Odoribacter in obesity (Etxeberria et al., “Pterostilbene-induced changes in gut microbiota composition in relation to obesity” Mol Nutr Food Res. 2017 61). However, it is common technical knowledge that the gut microbial diversity changes with diet (Rachel Feltman “The Gut's Microbiome Changes Rapidly with Diet” Scientific American, Dec. 14, 2013, https://www.scientificamerican.com/article/the-guts-microbiorne-changes-diet/, accessed 30 Aug. 2019) and little is known about the effect of pterostilbene in modifying gut microbial diversity after intake of foods rich in quaternary amines, specifically carnitine which is the source of TMAO. The present invention discloses the effect of pterostilbene in reducing the levels of circulating TMAO by a) modifying gut microbial diversity after consumption of foods rich in quaternary amines, and b) inhibiting the activity of FMO3.

It is the principle object of the invention to disclose a composition comprising pterostilbene for reducing the circulating levels of TMAO in mammals.

It is another object of the invention to disclose a composition comprising pterostilbene for modifying gut microbial diversity altered by foods rich in quaternary amines.

It is yet another object of the invention to disclose a composition comprising pterostilbene for inhibiting the activity of liver monooxygenase enzyme FMO3.

The present invention solves the above mentioned objectives and provides related advantages.

### SUMMARY OF THE INVENTION

In a preferred embodiment, the present invention discloses a method for reducing the levels of circulating TMAO in mammals, comprising step of administering a composition comprising pterostilbene to mammals in need of such reduction.

Specifically, the invention discloses a method for reducing the levels of circulating TMAO in mammals using a composition comprising pterostilbene by modifying gut microbial diversity altered by foods rich in quaternary amines and by inhibiting liver mono-oxygenase enzyme FMO3.

Other features and advantages of the present invention will become apparent from the following more detailed description, taken in conjunction with the accompanying images, which illustrate, by way of example, the principle of the invention.

### BRIEF DESCRIPTION OF THE FIGURES

The patent or application file contains at least one drawing, executed in color. Copies of this patent or patent application publication with color drawing(s) will be provided by the office upon request and payment of the necessary fee.

FIG. 1A is the graphical representation showing the results of polymerase chain reaction used to quantify expression levels of inflammatory gene VCAM-1. Values are given as mean±SE. ND indicates normal diet; Car, carnitine; Pte, pterostilbene; Abs, antibiotic cocktail.

FIG. 1B is the graphical representation showing the results of polymerase chain reaction used to quantify expression levels of inflammatory gene TNFα. Values are given as mean±SE. ND indicates normal diet; Car, carnitine; Pte, pterostiIbene; Abs, antibiotic cocktail.

FIG. 1C is the graphical representation showing the results of polymerase chain reaction used to quantify expression levels of e-selectin. Values are given as mean±SE. ND indicates normal diet; Car, carnitine; Pte, pterostilbene; Abs, antibiotic cocktail.

FIG. 2A is the graphical representation showing the Plasma TMAO levels of female B6 mice were measured using LC-MS/MS. Values are given as mean±SD, n=3 for each group. ND indicates normal diet; Car, carnitine; Pte, pterostilbene; Abs, antibiotic cocktail.

FIG. 2B is the graphical representation showing the Plasma carnitine levels of female B6 mice were measured using LC-MS/MS. Values are given as mean±SD, n=3 for each group. ND indicates normal diet; Car, carnitine; Pte, pterostilbene; Abs, antibiotic cocktail.

FIG. 3A is the graphical representation showing the heat map of 16S rRNA gene sequencing analysis of cecal microbiota at the genus level. ND indicates normal diet; Car, carnitine; Pte, pterostilbene; Abs, antibiotic cocktail.

FIG. 3B is the graphical representation showing Erysipelotrichia (Turibacter) proportions, significantly changed by carnitine supplementation. ND indicates normal diet; Car, carnitine; Pte, pterostilbene: Abs, antibiotic cocktail.

FIG. 3C is the graphical representation showing partial least squares discriminant analysis (PLS-DA) demonstrates distinct cecal microbial composition among groups. Each data point represents one sample and each color represents each group (percent variation explained by each PLS is shown in parentheses). ND indicates normal diet; Car, carnitine; Pte, pterostilbene; Abs, antibiotic cocktail.

FIG. 4 is the graphical representation showing the expression levels of hepatic FMO3 mRNAs determined by RT-qPCR and normalized to GAPDH. Values are given as mean±SD, n=3 for each group. ND indicates normal diet; Car, carnitine; Pte, pterostilbene; Abs, antibiotic cocktail.

### DESCRIPTION OF THE MOST PREFERRED EMBODIMENTS

In a most, preferred embodiment, the invention discloses a method for normalizing elevated levels of circulating Trimethylamine-N-oxide in mammals, comprising step of administering a composition comprising pterostilbene to mammals in need of such reduction. In a related aspect, the Trimethylamine-N-oxide levels are elevated after consumption of foods rich in quaternary amines. In a related aspect, the quaternary amines are selected from the group comprising of choline, carnitine, glycine betaine (GBT), and phosphatidylcholine and lecithin. In another related aspect, the quaternary amine is carnitine. In yet another related embodiment, the foods rich in quaternary amines are selected from the group comprising of meat, sea food, poultry, cooked green vegetables such as brussels sprouts and broccoli, legumes such as soybeans, kidney beans, and black beans, and milk.

In a related aspect the reduction in Trimethylamine-N-oxide levels by a composition comprising pterostilbene is brought about by a) modifying the gut microbial diversity, altered by consumption of foods rich in quaternary amines and b) inhibition of liver mono-oxygenase enzyme FMO3. In another related aspect, the invention also discloses a method for modifying gut microbial diversity, wherein the gut microbial phyla, modified by pterostilbene are selected from the group comprising of Bacteroidetes, Deferribacteres, Firmicutes, Proteobacteria and Verrucomicrobia. In another related aspect, the gut microbial phyla modified by pterostilbene are selected from the group comprising of Bacteroidetes and Firmicutes. In another related aspect, the genus of gut microbiota modified by pterostilbene is selected from the group comprising of Enterobacter, Escherichia, Ruminococcaceae, Ruminiclostridium, Alistipes, Lachnospiraceae, Mucispirillum, Bacteroides, Marvinbryantia, Lactobacillus, Prevotellaceae, Blautia, Turibacter, Intestinimonas, Eubacterium, Oscillibacter, Lachnospiraceae, Lachnoclostridium, Ruminococcus, Anaerotruncus, Lachnospiraceae, Romboutsia, Parabacteroides, Butyricicoccus, Roseburia, Ruinococcaceae, and Akkermansia. In another related aspect, the genus of gut microbiota modified by pterostilbene is selected from the group comprising of Bacteroides and Turibacter.

In another related embodiment, the composition comprising pterostilbene is formulated with pharmaceutically/nutraceutically acceptable excipients, adjuvants, bioavailability enhancers, antioxidants, diluents or carriers and administered orally in the form of tablets, capsules, syrups, gummies, powders, suspensions, emulsions, chewables, candies or eatables.

The aforesaid most preferred embodiments incorporating the technical features and technical effects of instant invention, are explained through illustrative examples herein, under.

### EXAMPLES

### Example 1: Pterostilbene for Reducing Trimethylamine-N-Oxide

Animal Model and Treatment Protocol

Six-week-old female C57BL/6 (B6) mice were purchased from BioLASCO (Taipei, Taiwan) and were acclimatized for 1 week. Mice were fed ad libitum with experimental diets, LabDiet Rodent 5001. The animal use protocol listed below has been reviewed and approved by the Institutional Animal Care and Use Committee (NTU105-EL-00115). In the experiment, mice were randomly divided into four groups: normal diet, 1.3% carnitine water, 1.3% carnitine water with 0.05% pterostilbene diet (around 250 mg/kg/day), and 1.3% carnitine water with antibiotic cocktail for 6 weeks. Mice received a cocktail of oral antibiotics (vancomycin 500 mg/L, metronidazole 1 g/L, neomycin 1 g/L, and ampicillin 1 g/L in drinking water) for 6 weeks. Antibiotic cocktail showed to suppress commensal gut microbiota and was given via gastric gavage every 12 hours. At the end of the six-week study, mice were euthanized by CO2 asphyxiation.

RNA Extraction and Reverse Transcription

Hepatic and aortic RNA was extracted using TRIzol reagent. Phase separation was performed by adding 200 μl chloroform/1 mlTRIzol. Following centrifugation, the aqueous phase (top) was transferred to a new tube. Added 500 μl isopropanol to the new tube and incubated at room temperature for 10 minutes for RNA precipitation. 75% ethanol was used to remove organic reagents and salts. Resuspend the pellet in ddH2O.

Quality of RNA and RNA concentrations were then quantified using NanoDrop 1000 spectrophotometer. Each RNA sample was then reverse-transcribed to cDNA using 1 μg of RNA. Reverse transcription was performed using the SensiFAST™ cDNA synthesis kit including random hexamers and oligo (dT)s in a 20 μl total reaction volume following manufacturer's instruction. Briefly, reverse transcription was carried out for 15 min at 42° C. followed by heating at 85° C. for 5 min to inactivate the enzyme. The resulting cDNA was stored at −20° C. for long term storage and used as a polymerase chain reaction (PCR) template.

Quantitative Real-Time Polymerase Chain Reaction (qPCR)

mRNA levels were determined by quantitative real-time PCR usingStepOnePlus Real-Time PCR system and SYBR Green master mix as compared to constitutively expressed gene (GAPDH) using the relative quantification method (ΔΔCt), Primer sequences for SYBR Green reactions were designed and synthesized by Mission Biotech. The list of primer sequences is as below:

TABLE 1
Primer sequences

| | Primer sequence | Product |
|---|---|---|
| Gene name | (5′-3′) | size (bp) |
| FMO3 | TGCTGCAGAACTCAGCCATGTAGCTC | 183 |
| | ATCTGCCTTGTGTACCACCAGTC | |
| TNF-α | AGTGACAAGCCTGTAGC | 202 |
| | TGAGGAGCACGTAGTCG | |
| VCAM-1 | AGTTGGGGATTCGGTTGTTC | 107 |
| | CATTCCTTACCACCCCATTG | |
| E-selectin | CCAGAATGGCGTCATGGA | 89 |
| | TAAAGCCCTCATTGCATTGA | |
| GAPDH | TCAACGGCACAGTCAAGG | 126 |
| | ACTCCACGACATACTCAGC | |

Quantification of Plasma Carnitine and TMAO Levels

LC-MS/MS was used to quantify plasma L-carnitine and TMAO in positive MRM mode, with the instrument water UPLC system (Acquity, Waters, Mildford, Mass., USA) and triple quadrupole mass spectrometer (TQS, Waters Micromass, Manchester, UK). The separation was performed using Agilent ZORBAX NH2 Column (5 μm, 4.6 mm×250 mm) and the column was thermostated at 30° C. Mobile phase A was composed of 10 mM NaOAc and 0.6% acetic acid in ACN and mobile phase B was 10 mM NaOAc and 0.6% acetic acid in water. The gradient profile is shown in Table 2.

TABLE 2
Gradient profile used in LC-MS/MS method

| Gradient | Flow rate | Mobile phase | Mobile phase |
|---|---|---|---|
| time(min) | (mL/min) | A (%) | B (%) |
| Initial | 0.800 | 10.0 | 90.0 |
| 0.50 | 0.800 | 10.0 | 90.0 |
| 1.00 | 0.800 | 20.0 | 80.0 |
| 2.50 | 1.000 | 45.0 | 35.0 |
| 3.00 | 1.000 | 60.0 | 40.0 |
| 4.00 | 1.000 | 60.0 | 40.0 |
| 4.45 | 1.000 | 70.0 | 30.0 |

The MS parameters were set as follow: 100° C. for the source temperature, 650° C. for the desolvation temperature, 2 L/h for the cone gas flow, 600 L/h for the desolvation gas flow and 1500 V for the capillary voltage. Cone voltage was set a 10 V. Concentrations of each analyte in samples were determined by calibration curves.

Microbiota Analysis by Next Generation Sequencing

Microbial community composition was assessed by pyrosequencing 16S rRNA genes derived from mouse cecum (n=3 for each group). Total DNA was isolated and purified using InnuSPEED Stool DNA kit according to the manufacturer's instructions. The quality of DNA samples was assessed by NanoDrop 1000 spectrophotometer. DNA samples were sent to Biotools Co., Ltd.for 16S rRNA gene amplification, sequence library construction and sequencing. The V3-V4 regions of bacterial 16S rDNA were amplified using PCR technology. The library DNA was sequenced by IlluminaHiSeq2500 platform, and paired-end reads (250 bp) were generated.

Statistical Analysis

Quantitative data are presented as mean±standard deviations (SD). Statistical analysis was conducted with one-way analysis of variance (ANOVA) using SPSS 12.0. A p-value was less than 0.05 considered as statistically significant, and the Duncan's multiple range post hoc test was applied if the p-value was less than 0.05. Graphs were created using SigmaPlot 12.5 software.

Results

Six-week-old C57BL/6 (B6) mice with a similar initial weight were randomly divided into four experimental groups as follows: receiving normal diet (ND). 1.3% carnitine water (Car), carnitine water with 0.05% pterostilbene diet (Car+Pte), and carnitine water with antibiotic cocktail (Car+Abs). As metronidazole gives such a foul taste to the drinking water that mice refrain from drinking, Car+Abs group mice were provided oral, antibiotic concoction (including vancomycin 500 mg/L, metronidazole 1 g/L, neomycin 1 g/L, and ampicillin 1 g/L in drinking water) for 6 weeks to deplete their gut microbiota. After 6 weeks, the mice were euthanized by CO2 asphyxiation for further mechanistic investigation.

Analysis of Vascular Inflammation Gene Expression in Aortas and Evaluation of Plasma Carnitine and TMAO Levels

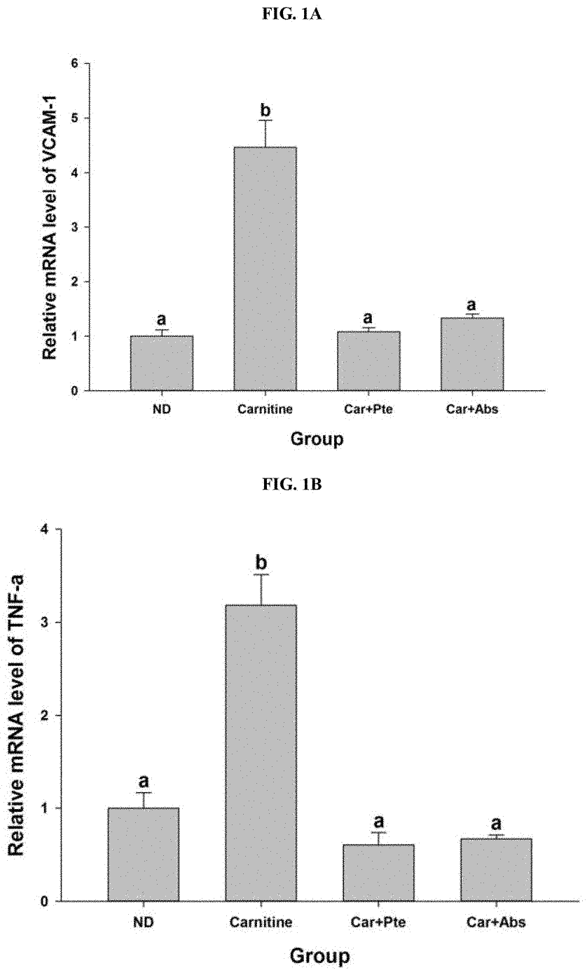

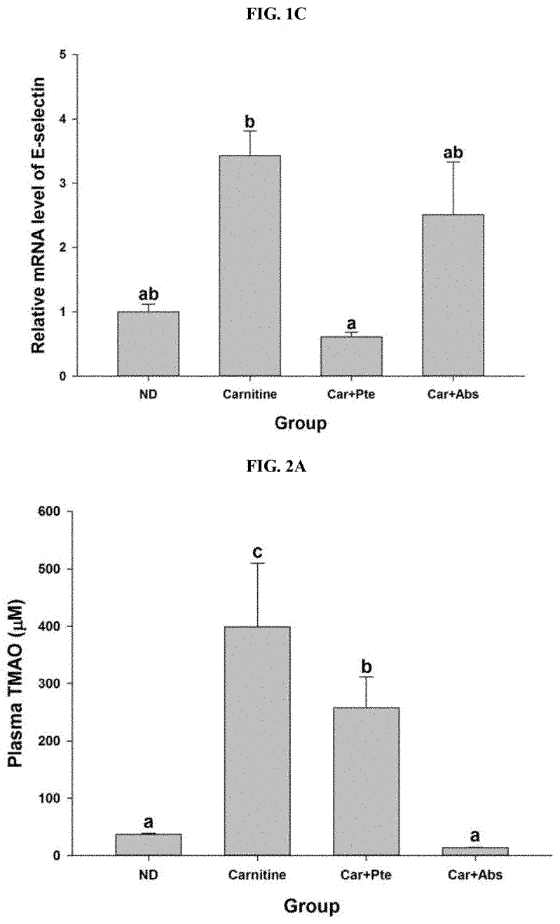

Carnitine group showed significantly enhanced expression of VCAM-1, TNF-α and E-selectin compared to ND group (FIGS. 1A, 1B and 1C). Vascular inflammation gene expression in Car+Pte group was significantly lowered as compared to Carnitine group and was similar to ND group. Car+Abs group lowered VCAM-1 and TNF-α.

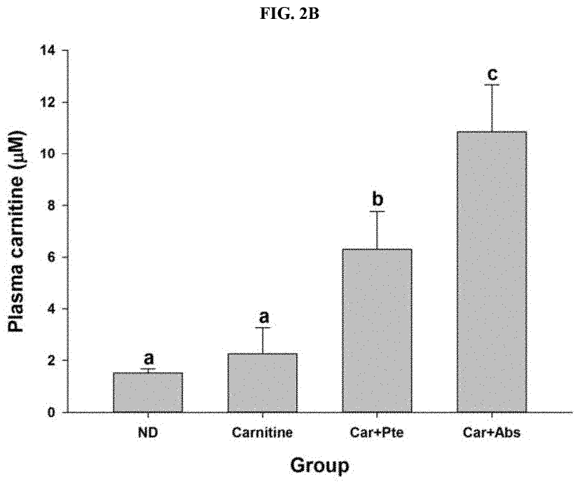

Circulating TMAO was significantly higher in Carnitine group compared to ND group in up to 8 folds increased in TMAO level (FIG. 2A). However, plasma TMAO level in Car+Abs group dramatically decreased as the fact that TMAO production from carnitine is inducible by host gut microbiota metabolism. With antibiotic administration, TMAO producing capacity is almost completely depleted. In Car+Pte group, plasma TMAO also significantly decreased compared to Carnitine group although the suppressions of TMAO elevation do not similar to that of the ND group. Plasma carnitine level of Car+Abs group is highest among groups as there is no gut microbial metabolism of carnitine to TMAO in antibiotic-treated mice (FIG. 2B). Plasma carnitine in Carnitine group is similar to ND group. Notably, pterostilbene increased circulating carnitine and decreased plasma TMAO levels. This result suggested that pterostilbene might suppress gut microbiota metabolic activity for metabolizing carnitine to TMAO and improve carnitine absorption.

Analysis of Intestinal Microbiota Composition

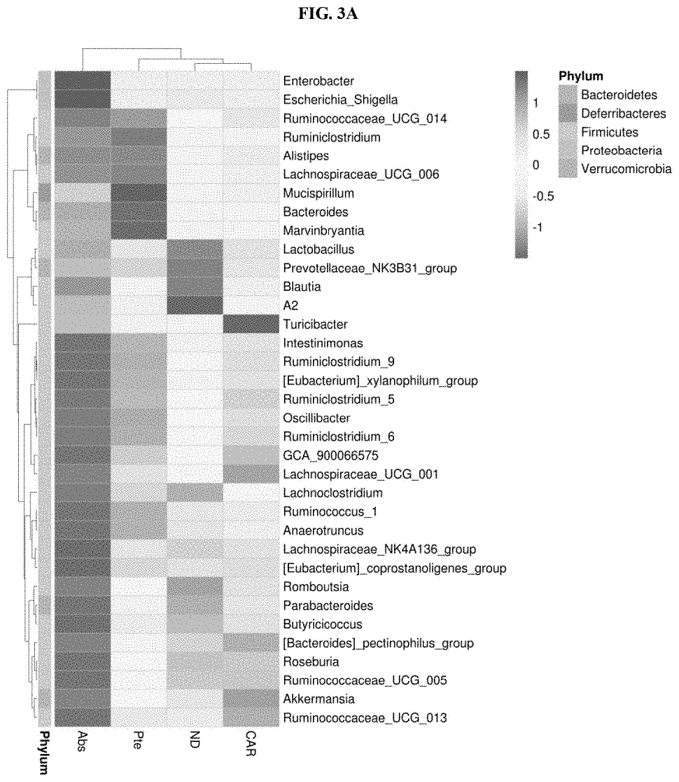

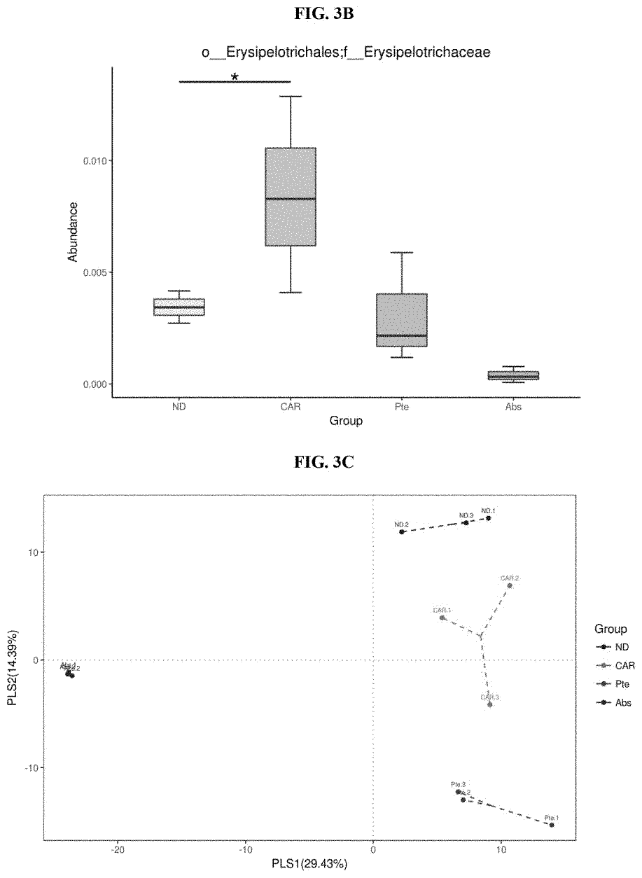

Genus-level analysis, showed that there is an increase in relative abundance of Turibacter, which belongs, to Erysipelotrichia, in Carnitine group (FIGS. 3A and 3B). Pterostilbene increased the relative abundance of Bacteroides compared to Carnitine group (FIG. 3A), Bacteroides is reported to be negatively associated with plasma TMAO (Chen et al., “Resveratrol Attenuates Trimethylamine-N-Oxide (TMAO)-Induced Atherosclerosis by Regulating TMAO Synthesis and Bile Acid Metabolism via Remodeling of the Gut Microbiota” MBio. 2016 7, e02210-e02215). Notably, Car+Pte group decreased Erysipelotrichia proportion compared to Carnitine group. These findings suggested that pterostilbene can manipulate gut microbiota altered by carnitine feeding.

PLS-DA plot showed that PLS 1 and PLS 2 explained 29.43% and 14.39% of variation of gut microbiota composition, respectively (FIG. 3C). This finding indicates that pterostilbene might leads to a novel therapeutic intervention for reversing vascular inflammation, and attenuating atherosclerosis through gut microbiota remodeling and decreasing TMAO-producing capacity.

Analysis of Hepatic Enzyme FMO3 mRNA Expression Level

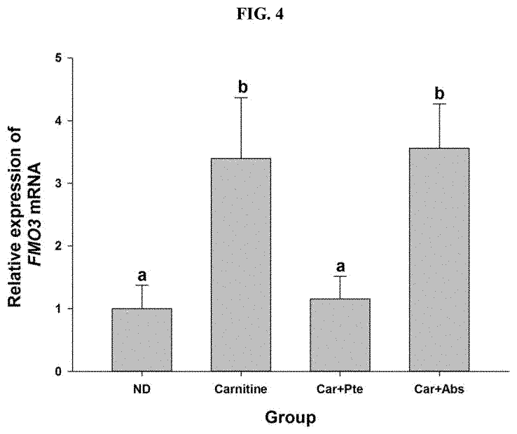

Carnitine group caused an approximately three-fold increase of hepatic FMO3 mRNA expression level compared to ND group. However, there was a significant reduction of hepatic FMO3 mRNA levels were observed in pterostilbene compared to Carnitine group (FIG. 4). This might be the reason why Car+Pte group decreased plasma TMAO. Antibiotic administration suppressed secondary bile acids production and caused a buildup of primary bile acids like cholic acid as antibiotics administration group showed an increase of FMO3 mRNA level similar to Carnitine group.

### Example 2: Compositions/Formulations Containing Pterostilbene

Given that pterostilbene is useful in decreasing the level of circulating TMAO, by modifying the gut microbial diversity it can be used as a dietary supplement to alleviate the effects the TMAO, specifically after consumption of foods rich (e.g. red meat) in quaternary amines, especially carnitine. It can be effectively blended in different compositions/formulations that can be consumed after eating carnitine rich foods to alleviate their potential ill-effects.

In a related aspect, one or more anti-oxidants and anti-inflammatory agents are selected from the group consisting of but not limited to, vitamin A, D, E, K, C, B complex, rosmarinic acid, Alpha Lipoic Acid, oxyresveratrol, Ellagic Acid, Glycyrrhizinic Acid, Epigallocatechin Gallate, plant polyphenols, Glabridin, moringa oil, oleanolic acid, Oleuropein, Carnosic acid, urocanic acid, phytoene, lipoid acid, lipoamide, ferritin, desferal, billirubin, billiverdin, melanins, ubiquinone, ubiquinol, ascorbyl palmitate, Mg ascorbyl phosphate, ascorbyl acetate, tocopherols and derivatives such as vitamin E acetate, uric acid, α-glucosylrutin, calalase and the superoxide dismutase, glutathione, selenium compounds, butylated hydroxyanisole (BHA), butylated hydroxytoluene (BHT), sodium metabisulfite (SMB), propyl gallate (PG) and ammo acid cysteine.

In another related aspect, one or more bioavailability enhancers are selected from the group, but not limited to, piperine, tetrahydropiperine quercetin, Garlic extract, ginger extract, and naringin.

Tables 2-4 provide illustrative examples of formulations containing pterostilbene.

TABLE 2
Premix containing pterostilbene

Active Ingredients
Pterostilbene (50-500 mg)
β-glucogallin and mucic acid gallates, Bacillus coagulans MTCC 5856,
Fenumannans, Triphala Aquasol, 20% Gingerols,
Excipients
Maltodextrin, Citric Acid, Malic Acid, Sucralose, Lime, Spearmint and
Mangoginger flavours and artificial Mint Flavour, Cumin powder, Black
Salt powder, Asafoetida
Directions for use: Dissolve the required premix in 200-300 ml water and mix well before use

TABLE 3
Pterostilbene Tablet

Active Ingredients
Pterostilbene (50-500 mg)
Excipients
Microcrystalline cellulose, Hypromellose, Croscarmellose Sodium,
Colloidal silicon dioxide, Magnesium stearate

TABLE 4
Pterostilbene Capsule

Active Ingredients
Pterostilbene (50-500 mg)
Excipients
Microcrystalline cellulose, Croscarmellose Sodium, Magnesium
stearate

Table 5 provides illustrative example of a chewable gummy composition containing Pterostilbene

TABLE 5
Pterostilbene Gummy composition

Active Ingredients
Pterostilbene (50-500 mg), Pectin, Glucose corn syrup
Excipients
Citiric acid, Lactic acid, Lemon peel oil (flavor), DL Tartaric acid,
refinated sugar

The above formulations are merely illustrative examples, any formulation containing the above active ingredient intended for the said purpose will be considered equivalent.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

We claim:

1. A method for normalizing elevated levels of circulating Trimethylamine-N-oxide in mammals, comprising step of administering a composition comprising pterostilbene to mammals in need of such reduction.

2. The method as in claim 1, wherein the Trimethylamine-N-oxide levels are elevated after consumption of foods rich in quaternary amines.

3. The method as in claim 1, wherein the reduction in Trimethylamine-N-oxide levels by a composition comprising pterostilbene is brought about by a) modifying the gut microbial diversity, altered by consumption of foods rich in quaternary amines and b) inhibition of liver mono-oxygenase enzyme Flavin Mono Oxygenase 3.

4. The quaternary amines as mentioned in claim 2, wherein the quaternary amines are selected from the group consisting of choline, carnitine, glycine betaine, and phosphatidylcholine and lecithin.

5. The quaternary amines as mentioned in claim 2, wherein the quaternary amine is carnitine.

6. The foods rich in quaternary amines as mentioned in claim 2, wherein the said foods are selected from the group consisting of meat, sea food, poultry, cooked green vegetables such as brussels sprouts and broccoli, legumes such as soybeans, kidney beans, and black beans, and milk.

7. The method for modifying gut microbial diversity as mentioned in claim 3, wherein the gut microbial phyla, modified by pterostilbene are selected from the group consisting of Bacteroidetes, Deferribacteres, Firmicutes, Proteobacteria and Verrucomicrobia.

8. The method for modifying gut microbial diversity as mentioned in claim 3, wherein the gut microbial phyla modified by pterostilbene are selected from the group consisting of Bacteroidetes and Firmicutes.

9. The method for modifying gut microbial diversity as mentioned in claim 3, wherein genus of gut microbiota modified by pterostilbene is selected from the group consisting of Enterobacter, Escherichia, Ruminococcaceae, Ruminiclostridium, Alistipes, Lachnospiraceae, Mucispirillum, Bacteroides, Marvinbryantia, Lactobacillus, Prevotellaceae, Blautia, Turibacter, Intestinimonas, Eubacterium, Oscillibacter, Lachnospiraceae, Lachnoclostridium, Ruminococcus, Anaerotruncus, Lachnospiraceae, Romboutsia, Parabacteroides, Butyricicoccus, Roseburia, Ruinococcaceae, and Akkermansia.

10. The method for modifying gut microbial diversity as mentioned in claim 3, wherein genus of gut microbiota modified by pterostilbene is selected from the group consisting of Bacteroides and Turibacter.

11. The method as in claim 1, wherein the composition comprising pterostilbene is formulated with pharmaceutically/nutraceutically acceptable excipients, adjuvants, bioavailability enhancers, antioxidants, diluents or carriers and administered orally in the form of tablets, capsules, syrups, gummies, powders, suspensions, emulsions, chewables, candies or eatables.
